(19) United States
(12) Patent Application Publication
(10) Pub. No.: US 2008/0262289 A1
(43) Pub. Date: Oct. 23, 2008

(54) Blood Pump Having A Passive Non-Contacting Bearing Suspension

(76) Inventor: Michael P. Goldowsky, Myrtle Beach, SC (US)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 12/100,686

(22) Filed: Apr. 10, 2008

Related U.S. Application Data
(63) Continuation-in-part of application No. 10/845,183, filed on May 14, 2004, now abandoned.
(60) Provisional application No. 60/473,587, filed on May 28, 2003.

(51) Int. Cl. A61M 1/10

(52) U.S. Cl. 600/16; 415/118; 415/120; 415/171.1

(57) ABSTRACT

There is provided a non-contact, passively suspended blood pump that includes (a) a housing; (b) a pump rotor within the housing, wherein the pump rotor has a first end and a second end, and an axis of rotation; (c) a first axial thrust bearing across a first axial gap, between the first end and the housing, that axially suspends the first end; (d) a second axial thrust bearing across a second axial gap, between the second end and the housing, that axially suspends the second end; (e) a first radial hydrodynamic bearing that radially suspends the first end; and (f) a second radial hydrodynamic bearing that radially suspends the second end. Determining pump differential pressure by monitoring rotor axial position allows automatic physiologic control.

### BACKGROUND OF THE INVENTION

1. Field of the Invention

This invention relates to axial or centrifugal turbo blood pumps and more particularly to such blood pumps whose rotor is passively suspended, for measuring left ventricular assist devices (LVAD) differential pressure.

2. Description of the Related Art

First generation blood pumps utilized and still utilize flexible pumping ventricles in contact with blood. They have no blood immersed bearings and not prone to thrombosis. An advantage is their inherent pulsating flow. For one disadvantage they are too large for competitive use as LVADs (Left Ventricular Assist Devices). Newer second-generation turbo pumps have a high rpm impeller like the Jarvik 2000 and Micromed axial flow pumps. They are much smaller but have contacting bearings that suspend the rigid motor. Most second-generation pumps are the larger centrifugal type. In axial and centrifugal turbo pumps, bearing contact results in undesirable clot formation either inside or around the periphery of the bearings. Such pumps are not suitable for long term reliable use. A major improvement in turbo pumps has been the relatively recent improvement of incorporating non-contacting bearings to eliminate the main remaining problem of thrombosis. These are known as third generation turbo pumps. Hydrodynamic blood immersed bearings and magnetic bearings are currently state of the art and are in development. No third generation pump presently has U.S. FDA approval for general use. The Incore 1 (trademark) (Berlin Heart Corp., Berlin, Germany) recently received EU Seal of Approval for marketing in Europe. It has a fully magnetically suspended rotor.

Goldowsky U.S. Pat. No. 6,527,699 titled: “Magnetic Suspension Blood Pump” discloses a similar and smaller third generation axial flow miniature turbo pump, whose rotor is suspended both radially and axially using fringing ring non-contacting magnetic bearings. The bearing is radial passive and unstable axially. An active control system is used to stabilize the bearing axially and absorb rotor axial pressure forces. Measuring rotor axial position by implementing virtually zero power feedback control allows measuring pump differential pressure (on which physiologic control can be based). This is a “smart” magnetic bearing.

A disadvantage of third generation pumps is the fact that an electronic control system is required to stabilize the magnetic bearing. This requires space that is at a premium in implanted devices, particularly in children and small adults. Pump size is to be minimized and this also reduces infection. Control electronics contribute to unreliability not to mention their additional cost. In the above Goldowsky patent, back-up mechanical axial thrust bearings or pins are provided should the control system fail. These are not needed in the instant invention because there is no bearing electronics to fail. The elimination of an actively controlled rotor with its control system in the instant invention is clearly advantageous and extends the present art. Consequently, the primary purpose of the instant invention, is to devise a passive rotor suspension for use in axial as well as centrifugal turbo blood pumps and to provide inherent capability to measure LVAD differential pressure. This shall be called a fourth generation pump.

Some turbo pumps employ radial hydrodynamic journal bearings that eliminate contact. An example is the Cleveland Clinic (Cleveland, Ohio) centrifugal “Coraid” (trademark). The axial thrust on the impeller is absorbed using the passive magnetic-reluctance force of the motor. There is no teaching in their published or patent literature to monitor impeller axial position as a measure of differential pressure.

Some pumps use a radial load capacity hydrodynamic journal bearing like the above, but with an axial hydrodynamic thrust bearing or a non-desirable contacting thrust bearing to hold axial forces. An example of one with all hydrodynamic bearings is the VentraAssist (trademark) centrifugal, (Ventracor Ltd, Sydney, Australia). It does not measure rotor axial position or differential pressure. Radial magnetic bearing pumps have attempted to employ axial hydrodynamic thrust bearings. Hydrodynamic thrust bearings pose difficult design constraints in order not to damage blood. Their small gap of typically a mil (0.001″) characteristically possesses high blood shear, which causes hemolysis with potential clotting. Such small gaps are difficult to adequately wash out to eliminate thrombosis. If a hydrodynamic thrust bearing functions, its axial deflection is so small (with typical differential pressures of 0-150 mmhg) that deflection cannot be reliably measured to accurately determine differential pressure. Also, the stiffness properties of the bearing are dependent on blood viscosity so its calibration is not constant and changes. These disadvantages are overcome in the instant invention using a passive magnetic thrust bearing that has substantial axial deflection that can be easily and accurately measured independent of blood properties.

Full, non-contacting magnetic suspensions have been a successful approach in both axial and centrifugal pumps. For example, the University of Utah (Salt Lake City, USA) HeartQuest (trademark) centrifugal pump employs passive radial magnets to support the impeller. However, the impeller is unstable axially so active electronic axial control is used. They do not teach monitoring rotor axial position to determine differential pressure. An example of a successful axial flow pump is the Incore 1 (trademark) (Berlin Heart Corp., Berlin, Germany). It is similar magnetically to the Goldowsky patent cited above, but is four times larger and does not claim to inherently obtain differential pressure.

Ernshaw's Law (circa 1800's) states that: “A rigid body cannot be totally magnetically suspended passively in all axes”. There must be at least one axis of instability and this axis requires active control for stabilization. The above pumps exemplify this. Fully magnetically suspended rotors that have active or passive type magnetic bearings are not the ultimate in simplicity and reliability because a control system is required with undesirable electronics.

### SUMMARY OF THE INVENTION

Accordingly, there are three primary objects of the present invention. One is to provide a totally passive, non-contacting rotor suspension (requiring no control electronics). Another is to provide an axial thrust bearing possessing sufficient deflection and having a passive restoring stiffness independent of blood properties to absorb rotor axial forces without contact. Thirdly, is the ability to easily measure rotor axial position to obtain LVAD differential pressure.

Other objects of the present invention are to provide a very small yet high force capability magnetic thrust bearing design that is axis symmetric. Another object of the thrust bearing is to shield its fields from interfering with the motor and to provide a linear restoring force characteristic. Another object is to provide unusually large radial and axial bearing gaps that possess low hemolysis (red blood cell damage) that can be easily washed out to eliminate thrombosis. It is another object to provide forced pressure washout of the hydrodynamic radial bearing to avoid stasis and clotting therein. Another object is to provide unidirectional high-pressure washout of the hydrodynamic bearing to eliminate blood stagnation and regurgitation in the bearing gap and at the bearing exit and inlet.

It is also an important object of this invention to reliably and accurately sense differential pressure by incorporating a “smart thrust bearing” (one that is a transducer as well as a bearing). Differential pressure can be the basis for creating not only pulsating flow to mimic the natural heart but to provide physiologic flow control responsive to exercise level. It is yet another object to accomplish physiologic control in a safe manner by sensing and avoiding adverse suction at the pump inlet based (at least in part) on pump differential pressure.

Another object is to advance the state of the art for turbo pumps designed for long term use (years) by minimizing the generation of micro-emboli. This phenomenon has not yet been addressed in the design of present art turbo pumps. It is also an object to improve the hydraulic efficiency of small axial flow turbo pumps by eliminating leakage of blood past the blade tips. A key object of this invention is to provide the ultimate in LVAD mechanical simplicity with minimal electronics, because simplicity enhances reliability. Simplicity also lowers manufacturing cost, which is desirable to satisfy mass markets.

These and other objects of the present invention are provided in a structure for a turbo blood-pump using passive non-contacting smart bearing suspension. The present invention creates a hybrid bearing, which is not a full magnetic suspension. Since the instant invention does not employ a purely magnetically suspended rotor, the rotor can be totally passive without violating Ernshaw's Law. The invention suspends the rotor radially using a mechanical hydrodynamic journal bearing; these type bearings are finding application in third generation pumps. The magnetic part of the instant suspension is only axial and a passive magnet is employed in this axis. For high quality of life for the patient, providing pulsating flow (to minimize thrombosis and to increase blood perfusion of organs) as well as physiologic flow rate control (responsive to exercise level) is highly desired. The passive axial thrust bearing stiffness of the instant magnet pairs allows tailored and substantial axial deflection of the rotor for accurate measurement (yet the bearing can absorb shock without contact). Monitoring rotor axial position by having a “smart bearing” is one that allows monitoring LVAD differential pressure on which one may, at least in part, base physiologic control. Use of differential pressure for control is claimed in the Goldowsky patent cited above. That this is a practical way to control turbo pumps is documented in a scholarly paper by Giridharan, et al, entitled Modeling and Control of a Brushless DC Axial Flow Ventricular Assist Device, ASAIO Journal volume 48, No. 3, 2002.

Thus, an embodiment of the present invention is a blood pump that includes (a) a housing; (b) a pump rotor within the housing, wherein the pump rotor has a first end and a second end, and an axis of rotation; (c) a first axial thrust bearing across a first axial gap, between the first end and the housing, that axially suspends the first end; (d) a second axial thrust bearing across a second axial gap, between the second end and the housing, that axially suspends the second end; (e) a first radial hydrodynamic bearing that radially suspends the first end; and (f) a second radial hydrodynamic bearing that radially suspends the second end.

### BRIEF DESCRIPTION OF THE DRAWINGS

FIG. 1 is a longitudinal cross section through a preferred cylindrical axial flow turbo pump.

FIGS. 2 and 2A are diagrammatic representations, respectively, an end view and a longitudinal cross section, of a plain axially magnetized magnet, and the magnetic field emanating from the two ends thereof.

FIGS. 3 and 3A show, respectively, an end view and a partial longitudinal cross sectional view, through a permanent magnetic thrust bearing with a flux shielding cup similar to what is shown in FIG. 1, but additionally with an inner fringe.

FIGS. 4 and 4A show, respectively, an end view and a longitudinal sectional view of a thrust bearing using a radial magnetized magnet or magnetic segments, without a flux shielding cup.

FIGS. 5 and 5A show, respectively, an end view and a partial longitudinal sectional view, of a still further alternative thrust bearing with fringing rings, but no flux shielding cup.

FIGS. 6 and 6A show, respectively, a longitudinal view and an end view, of a journal bearing, showing the bearing and an integral screw pump at one end thereof, with the length of the bearing portion being labeled “L”.

FIG. 7 is a diagrammatic representation, as a longitudinal cross sectional view, through the journal of a hydrodynamic bearing which is fed with blood to a central groove.

FIG. 8 is a partial longitudinal section through an axial flow pump similar to FIG. 1, but utilizes a contact free hydrostatic thrust bearing that employs an integrated screw pump.

FIG. 8A is a non-sectioned enlarged view of the screw thread shown in FIG. 8.

FIG. 9 shows typical characteristic screw pump flow curves of pressure versus flow, for the screw pump shown in FIG. 8.

FIG. 10 is a longitudinal cross-section through a pump that employs two hydrodynamic bearings.

FIG. 11 is a longitudinal cross-section through a pump, showing a routing of blood in a non-serial path for washing out gaps within the pump.

### DESCRIPTION OF THE INVENTION

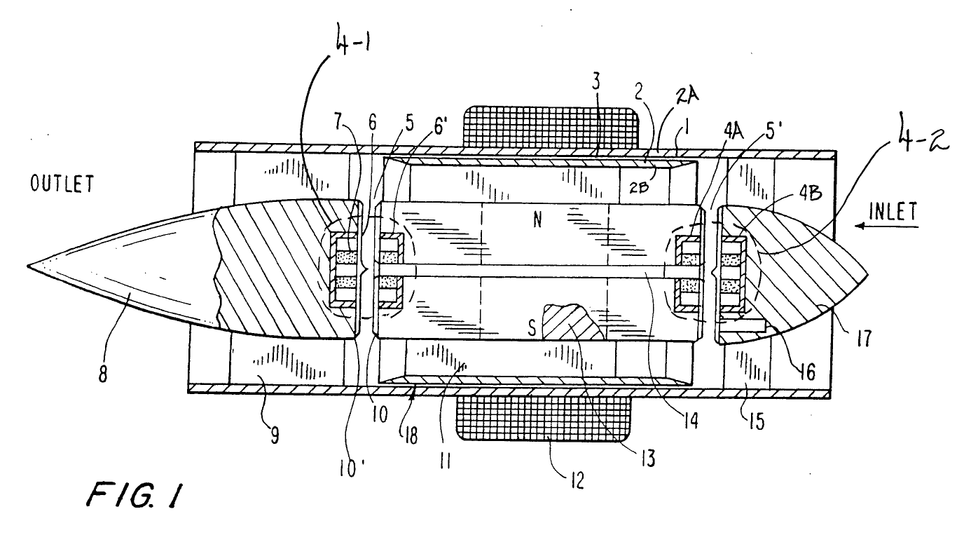

FIG. 1 is a longitudinal cross section through a preferred cylindrical axial flow turbo pump. FIG. 1 possesses limited detail, but has the essential elements of a similar axial flow pump described in FIG. 1 of Goldowsky U.S. Pat. No. 6,527,699. Only the major components of an axial flow turbo pump are shown in FIG. 1 in order to best describe the essential elements of the instant invention. An axial flow pump is illustrated and is not meant to be limiting. The same bearing suspension also applies to use in centrifugal turbo pumps. This is illustrated in the Goldowsky patent cited above.

A pump rotor is generally depicted as item 18. It includes helical impeller blades 11, a radial hydrodynamic bearing, i.e., a hydrodynamic bearing 2, axial magnet thrust bearings at each end (i.e., thrust bearings 4-1 and 4-2, each of which is shown in a dashed circle), thin windows 10 and 10′, brushless motor armature magnets 13, and a blood conduit 14 used to wash out axial rotor gaps at each end, i.e., gaps 5 and 5′.

Hydrodynamic bearing 2 runs a length of rotor 18 and includes a female portion 2A and a male portion 2B. Female portion 2A is also known as a journal.

Thrust bearing 4-2 is configured with a magnet 4A and a magnet 4B in a repulsive arrangement, acting across gap 5′ to counteract forces on rotor 18. Thrust bearing 4-1 is configured similarly to thrust bearing 4-2 in that it includes two magnets in a repulsive arrangement, acting across gap 5. Rotor 18 is suspended axially within housing 1 by the magnets of thrust bearings 4-1 and 4-2.

The concept of employing a central axial hole in the rotor to obtain gap washout flow is contained in Goldowsky U.S. Pat. No. 6,716,157 “Improved Magnetic Suspension Blood Pump”, issued Apr. 6, 2004. The magnet assemblies are hermetically sealed by thin (typically 0.010 thick) titanium or blood compatible sapphire window items 10. Hydrodynamic bearing 2 is separated from pump cylindrical housing 1 by a radial blood gap, i.e., a gap 3, whose combination forms a conventional hydrodynamic bearing. The rotor 18 has large axial blood gaps, i.e., gaps 5 and 5′, at each end. Brushless windings 12 surround a pump housing 1 and are commutated to rotate rotor 18.

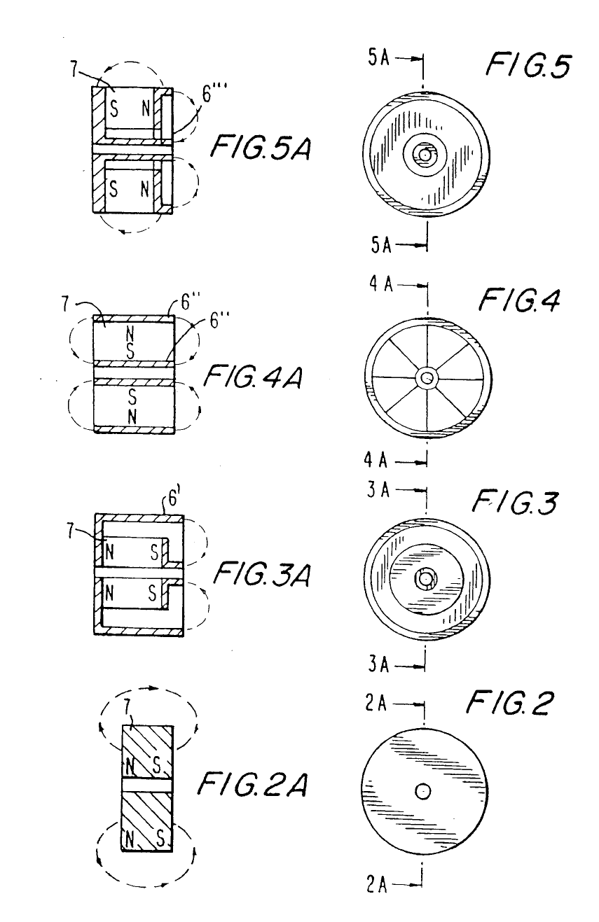

An outlet stator 8 has a plurality of fixed, flow straightening vanes 9 that are attached to pump housing 1. An inlet stator 17 is fixed to pump housing 1 by flow inlet vanes 15. Each stator 8, 17 houses a thrust bearing magnet assembly, e.g., inlet stator 17 houses magnet 4B. A central hole in the magnet of each of thrust bearings 4-1 and 4-2 is optional, but one is shown as it is preferable that each of the magnets be identical to its co-linear mate, e.g., magnet 4A is identical to magnet 4B. Thrust bearings 4-1 and 4-2 are preferably located in each end of rotor 18, as shown, to obtain bi-directional force capability. However, only one thrust bearing pair at the LVAD inlet is essential in order to hold differential pump pressure, which is directed toward the pump inlet. The symmetrical axial portion of the magnetic fields emanating from each pair of magnets oppose one another making a repelling force. Typical bucking fields are shown dotted in FIGS. 2-5 (the matching bearing mate is not shown). Rotor 18 is repelled in opposite axial directions at each end. This causes rotor 18 to find a stable central position under axial load. Thrust bearing 4 is axis symmetric to allow rotation and this occurs with very low eddy current losses due to symmetry.

A position sensor 16 uses rotor window 10 as a target to monitor rotor axial position. The preferred embodiment of sensor 16 is a miniature ultrasound probe (typically 2 mm in diameter) that operates from 5-15 MHz to have low attenuation in blood. It is insensitive to stray magnetic fields emanating from thrust bearings 4-1 and 4-2 and the motor. The probe tip in contact with blood may have diamond-like coating for blood compatibility. This embodiment of sensor 16 only requires a flat rigid window surface for its sound-beam to reflect from. In operation, the probe generates a short burst of sound (pulse), and then monitors the time of flight of the return pulse to determine target range. Range is calculated knowing sound velocity in blood, which is independent of blood viscosity and composition and gives stable performance. Sound velocity is close to that of pure water and nearly independent of blood type and properties since blood is mostly water. The sensor will not drift and is long term stable; a key requirement for long term blood pump use. This miniature ultrasound sensor is preferred and is claimed in Goldowsky U.S. Pat. No. 6,190,319 titled: “Self Calibrating Linear Position Sensor”.

Other types of sensors can also be employed such as eddy current, magnetic and capacitance types, but then sensor window and target materials used must be sensor compatible. Sensor 16 may be conveniently located on or off the pump axis provided it sees the target to measure position of rotor 18. An off axis location is depicted in FIG. 1 because this version of rotor 18 has an on axis blood washout hole, i.e., blood conduit 14, and the hole is an unsuitable target for such a small diameter transducer.

Blood conduit 14 may be a titanium tube for blood compatibility, with an inside diameter of about 1-2 mm. The choice of diameter will control the washout flow rate desired. Blood conduit 14 connects gaps 5 and 5′ in series with LVAD differential pressure (typically 100 mmhg). The resulting flow actively washes out both gaps 5 and 5′ with fresh blood to avoid thrombosis. There is a degree of centrifugal pressure generated in each gap 5 and 5′, but they are equal and oppositely directed so their effects cancel. Pump differential pressure is the main driving force that actively washes out gaps 5 and 5′. This is discussed in detail in Goldowsky U.S. Pat. No. 6,716,157, issued Apr. 6, 2004 entitled: “Improved Magnetic Suspension Blood Pump”.

Two important elements of the pump of FIG. 1 are the passive magnetic thrust bearings, i.e., thrust bearings 4-1 and 4-2, and the compact hydrodynamic rotary journal bearing, i.e., hydrodynamic bearing 2. Thrust bearing details are discussed first.

FIG. 1 shows an axially magnetized cylindrical magnet 7. It is preferably neodymium iron boron or samarium cobalt for very high strength and resistance to the demagnetization load placed on it by its paired opposing magnet. An iron or preferably high saturation vanadium-permendure cylindrical cup 6 surrounds magnet 7. Cup 6 retains the magnet's flux and substantially reduces stray fields which could otherwise interfere with the motor armature magnets 13 and vice versa, particularly in a miniature pump where the thrust bearings 4-1 and 4-2 are desired close to motor armature magnets 13 to minimize rotor length.

FIG. 3 shows the axial bucking field that emanates (shown dotted) from a similar geometry thrust bearing. In FIG. 1, cup 6 terminates at its outside diameter as a thin fringing ring 6′ which is used to focus the magnet flux to a high level (greater than attainable with a pure magnet alone). This flux produces a stronger repulsion force (proportional to gap flux density squared) in a much smaller size than is attainable with a pure magnet alone.

FIGS. 2 and 2A show the escaping or stray field emanating from a pure magnet. Its emanating axial field is weak and its axial force is small compared to that when a fringing ring is used. Fringing rings are ideally suited to minimize bearing size, which is required in a miniature pump.

FIG. 3 shows a thrust bearing geometry where a second fringing ring is located near to the center. The field emanates from the right end and when a matching bearing faces it, the two fields buck and repel. The closer they approach the higher the repulsion force which acts stably like a mechanical spring (negative stiffness) in the axial direction. However, unlike a mechanical spring, the pair is mutually radial unstable (positive stiffness). The rotor tries to “kick out” at each thrust bearing.

The magnet flux is concentrated higher in the fringe air gap of FIG. 3 than in the fringe air gap of thrust bearings 4-1 and 4-2, shown in FIG. 1. This is because the inner fringe has less cross sectional area than the outer, with the same amount of magnet flux, so its flux density is higher. The bearing of FIG. 3 is preferred over the configuration of thrust bearings 4-1 and 4-2 in FIG. 1 for performance but it requires an extra inner fringe.

Hydrodynamic bearing 2 is designed very stiffly in the radial direction to easily support the radial instability of the rotor's thrust bearings 4-1 and 4-2. Too large of an axial stiffness for thrust bearings 4-1 and 4-2 is not desired because axial deflection under load will be too small to easily measure and because their radial instability stiffness becomes undesirably high. For resonance reasons there could be constraints on the stiffness of hydrodynamic bearing 2 and the stiffness of thrust bearings 4-1 and 4-2.

These thrust bearing designs allow large gaps 5, and 5′ for ease of washout as well as large axial deflections. Use of large gaps 5 and 5′ allow absorbing the energy of large shock loads without contact even for relatively low axial stiffness. A typical gap 5, 5′ may be 0.3-3 mm and axial stiffness is easily tailored to be in the range of 20-200 lb/in.

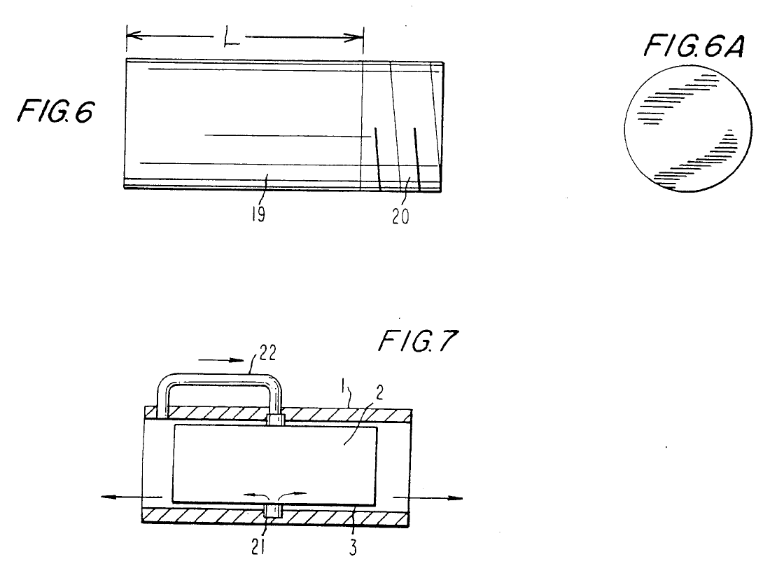

Another advantage of the iron cup used in bearings of FIGS. 1, 3 and 5 is excellent axial force linearity. Constant axial stiffness is desirable for rotor 18 for dynamic analysis. A linear force displacement characteristic is also desirable for the differential pressure transducer for easy calibration. A pair of plain magnets (a single magnet is shown in FIG. 2) is less desirable because it possesses a non-linear or exponential force. It is also much larger for the same stiffness and load capability and it has undesirable stray fields, detrimental in miniature and small pumps.

Whereas, FIGS. 1, 2, 2A, 3 and 3A show thrust bearings having a single axial magnetized magnet, the construction of the thrust bearing in FIGS. 4 and 4A utilizes several radial magnetized magnet segments. This is the conventional way to obtain a radial magnetized rare earth magnet (one piece radial magnets are just becoming available). Thin circular iron rings 6″ are located at the outside and inside diameters to capture the magnet's flux. The central hole (as in FIG. 1, blood conduit 14) allows blood to flow therethrough. This configuration is not magnetically efficient because leakage flux passes around the left end as shown and is wasted. This can be compensated for using a longer magnet.

FIGS. 5 and 5A show a thrust bearing that is similar in construction to thrust bearings 4-1 and 4-2 of FIG. 1, with an axial magnet, but does not have a complete cup to shield the field. Some field is wasted that axially shunts around the outside diameter as shown. An internal fringing ring 6′″ is added to focus the remaining flux to increase the force. Both outer and inner fringing rings contribute to repulsive force, the inner one generally contributes most of the axial force in FIGS. 3, 3A, 4, 4A, 5 and 5A.

Attention is now directed to the design of hydrodynamic bearing 2 in FIG. 1. The assembly of hydrodynamic bearing 2 consists of a thin cylindrical bearing sleeve rotating in a round journal, axially. The sleeve is separated from the journal by gap 3, which is blood immersed. The location of hydrodynamic bearing 2 is preferably outside of, and surrounds, impeller blades 11 in order to eliminate a blood gap at the O.D. of impeller blades 11, as well as not to occupy the space reserved for the motor magnets in the rotor hub.

Blade gaps in conventional axial flow pumps like the Jarvik 2000, must be made small to reduce back leakage past them or pump efficiency suffers. This is most important for the small pumps, especially those designed for children. However, gaps that are too small possess high shear and create hemolysis and can generate micro-emboli that lodge in end organs. The gap has been eliminated in the instant invention. The O.D. of impeller blade 11 is bonded to the I.D. of the sleeve of hydrodynamic bearing 2, or is made integral with it. No gap substantially improves pump hydraulic efficiency desirably reducing pump power. The housing bore is straight, rigid and ideally suited to being the journal.

Gap 3 is sized to be sufficiently large at the operating RPM of the pump to possess sub-hemolytic shear stresses. This minimizes blood hemolysis. However, gap 3 must be sufficiently small to produce the desired load capacity and radial stiffness over the LVAD's rpm operating envelope with variable blood viscosities. A larger gap 3 is desired for low power loss. These constraints have been met in the instant invention. Practical operating gaps 3 may fall in the range up to about 2 mm for axial flow pumps typically operating 7,000-20,000 rpm. The design of hydrodynamic bearing 2 (gap, diameter and length) must be compatible with the impeller blade 11 and operating rpm. Centrifugal turbo pumps on the other hand usually operate at much lower rpm (1,500-3,000). This necessitates use of a smaller gap 3.

For hydrodynamic bearing 2 to satisfactorily operate in blood, it is not sufficient to design just for load capacity. Stagnant areas must be insured against in gap 3 or blood will clot. To insure against clotting, it has been proven experimentally that supplying fresh blood under pressure to hydrodynamic bearing 2 to wash out gap 3 can eliminate regions of stagnation and formation of thrombus. FIGS. 6, 6A show a bearing that integrates a pressure generating screw pump 20 at one end, with a bearing portion 19 of length L. It is similar to those shown in Goldowsky U.S. Pat. No. 5,924,975 (“Linear Hydrodynamic Blood Pump”) and U.S. Pat. No. 6,436,027 (“Hydrodynamic Blood Bearing”). FIGS. 6, 6A are the preferred design of the instant bearing. Only a short section is needed for screw pump 20, particularly in axial flow turbo pumps because rpm is so high.

Screw pump 20 consists of a shallow helical screw thread of multiple starts. Blood is viscosity pumped along the thread grooves that are designed to generate pressure in excess of the outlet pressure of the LVAD (LVAD pressure typically 120 mmhg). The thread need only be a few mils deep allowing the sleeve of hydrodynamic bearing 2 to be thin to require little space. This creates continuous flow through hydrodynamic bearing 2, continuously washing it out. Screw pump 20 is preferably located near the pump inlet, which is at low pressure. Screw pump 20 pumps blood in one direction into the journal bearing gap, i.e., gap 3, of hydrodynamic bearing 2 and it exits at the bearing gap outlet to mix with LVAD blood. Since LVAD bulk blood is in the same direction as the gap flow, the two merge without regurgitation or back flow. Gap flow toward the LVAD outlet thereby eliminates stagnation regions to insure against thrombus formation at both of the inlet and the outlet of gap 3. The screw pump flow is made sufficiently large so blood does not become heated in hydrodynamic bearing 2 and to insure that a generous safety factor exists to wash out gap 3 under all operating conditions of the LVAD.

If a pressurizing pump (of some type) is not used to wash out the journal bearing gap one must take into account the phenomenon that a hydrodynamic bearing will try to pump fluid out both ends. If no fluid is supplied, the fluid will stagnate in the bearing, and if blood, it will clot. A fluid groove (or inlet holes) is conventionally used in bearings not designed for blood, and is located at the center of the journal. The groove allows fluid to be passively drawn in. In an automobile engine, it is supplied under pressure by an external oil pump. This is possible to implement in the instant invention.

One can use a sufficiently large tube that allows passage of fresh blood from the LVAD bulk flow (preferably at the higher pressure outlet) to the groove in the journal. This tube is shown schematically in FIG. 7 as a tube 22. A journal groove 21 typically subtends 360 degrees to make load capacity symmetrical in angular direction and to wash out all areas. Arrows at the bottom of groove 21 depict bearing flow out of groove 21 toward each end of the bearing where it exists. Tube 22 may alternatively be a hole in the wall of housing 1, but this is not practical for a thin wall, unless the wall is locally thickened. However, use of a central groove is not preferable compared to using an integral pump to supply pressurized-flow at the bearing end. A central groove undesirably reduces bearing load capacity by shortening bearing effective length L. This requires increasing bearing length to compensate. Grooves may also be prone to areas of stagnation that may clot with incipient bearing failure.

An integrated compact pump such as the screw pump located at the end of bearing portion 19 in FIG. 6, has been reliably used in blood with insignificant blood damage. This is an ideal solution to wash out the bearing. By placing screw pump 20 at one end of the bearing, the length of the bearing portion 19, labeled L, is maximized and is not broken up as when the screw is located at the center of the bearing. An end location provides a longer effective bearing length that maximizes load capacity and this allows use of a larger radial gap 3. A larger gap 3 has reduced blood shear stresses and less hemolysis. Hemolysis in an integrated hydrodynamic bearing/screw pump that was designed demonstrated a level for just the bearing that is 25 times lower (0.2) than is characteristic of turbo pumps (5 mg/dl).

A high pressure is needed to wash out gap 3 from one end so flow can be steady and sufficient. If no pressurizing pump is used to wash out gap 3, then one can use LVAD differential pressure alone (gap flow will be toward LVAD inlet) which is only about 100 mmhg. Gap 3 can be completely washed out using this relatively low pressure only if the internal hydrodynamic pressure pumping effect of the bearing is designed to be less. This internal pressure can be substantial. It is created by the rotating bearing and must be taken into account. Otherwise, a stagnant region will exist in the bearing and blood will clot causing bearing failure. Use of a pressurizing screw pump with sufficient pressure and flow avoids this, it is non-contacting, and it automatically operates with the bearing.

There is another undesirable phenomenon if only LVAD differential pressure is employed for bearing washout. The gap flow in the bearing is then toward the LVAD inlet, but the bulk LVAD flow is toward the outlet. Where the gap flow enters the journal bearing, fluid eddies will form with regions of stagnation, because the flow must reverse direction. This occurs at both ends of the bearing where blood enters and leaves the gap with the potential to create thrombus or clots and to generate micro-emboli. Alternatively, if blood is supplied to a central groove, gap flow opposes pump flow at the LVAD inlet, which can cause thrombus and micro-emboli.

Therefore, pump-less washout has hemodynamic reliability concerns. A unidirectional screw pump that directs bearing gap flow toward the LVAD outlet is superior. It eliminates this problem and will provide superior long term hemodynamic reliability.

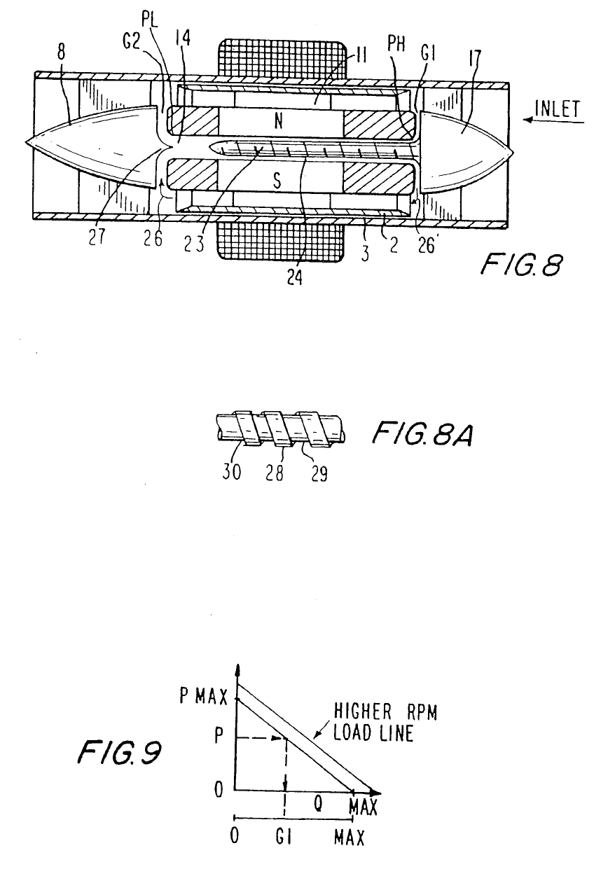

Having thus described the preferred invention, an alternate thrust bearing without magnets, is shown in FIG. 8. This is a hydro-static thrust bearing designed to hold axial load in one direction that is imposed by pump differential pressure. It has the desired stable negative axial stiffness possessing deflections that are much larger than in hydrodynamic thrust bearings. Its axial deflection under LVAD pressure loads is accurately measured using the preferred ultrasonic sensor discussed. A journal bearing with a hydrostatic thrust bearing designed to deliver cooling oil or water to electronic chips is disclosed in Goldowsky U.S. Pat. No. 5,713,670 titled: “Self Pressurizing Journal Bearing Assembly”. It is not tailored to work in blood but is similar.

The inlet stator 17 (or alternatively outlet stator) uses an attached stationary helical screw 23 that has a single or multiple start thread. It is provided enlarged in FIG. 8A for clarity. Thread lands 28 may be long or short. Thread groove 29 is typically flat-bottomed and corner radii 30 are employed for good washout. The screw is small enough in diameter not to appreciably take space from the motor armature magnets which can be made longer to compensate. Alternately, cylindrical passage 14 may have an internal helical screw thread; in this case a round pin is employed. Alternately, the screw shaft may be supported at both ends. The screw is located on the rotor center line with a radial gap 24 that never contacts by virtue of the stiffness of hydrodynamic bearing 2 whose gap is 3. Rotation of the rotor forces blood to flow and its pressure to increase along the screw helical groove (toward LVAD inlet). Blood enters the screw from large gap G2 in communication with LVAD bulk flow. There is little pressure drop through G2. So PL is nearly the same as rotor outlet pressure. This series flow washes out gap G2 as shown by arrows 26 and 26′. A pointed flow director 27, with a blend radius removes turbulence and allows the flow to smoothly enter central conduit 14. It also is a stop to insure that gap G2 is large at initial start up of the pump so the rotor initially lifts off axially toward LVAD inlet.

The tip of the screw may be an off-center point to divert flow non-symmetrically without a stagnation point, as may be the design of item 27. PL is increased by screw pump 23 to higher value PH at the screw outlet which enters gap G1. Gap G1 is much smaller than gap G2, thereby becoming the primary flow resistance on the screw pump. It is large enough to be subhemolytic. G1 forms a hydrostatic thrust bearing by virtue of the high pressure therein. Its thrust bearing area is equal to the end face area of the rotor hub (which is preferably the same at both ends as shown).

Gap G1 discharges blood 26′ to LVAD inlet pressure which is low. The (average pressure acting on rotor face G1 minus pressure PL on rotor face G2) multiplied by rotor hub area, is the net force of the thrust bearing acting toward the LVAD outlet. This balances the entire differential pressure force on the rotor (including its impeller blades). This unique combination or system of a screw pump and thrust bearing, automatically adjusts rotor gap G1 using hydraulic feedback until the thrust bearing force equals the externally applied rotor force. This automatic adjusting of rotor axial position is measured with a position sensor from which differential pressure is calculated by dividing by the effective area of the rotor.

A feedback system exists to drive rotor error position to zero because of the negative slope (pressure P versus flow Q) being inherent in the characteristic curves of screw pump 23. Schematic screw pump curves are shown in FIG. 9. The two “curves” are linear with the upper one being at higher rpm. These lines are parallel and linearly spaced apart as a function of rpm. When screw pump flow rate Q is a maximum value (Q max), zero pressure rise is generated and when Q is zero, maximum pressure rise is generated. The amount of flow Q at a given pressure P is shown dotted which gives the typical operating point; and so operating gap G1 results, it being shown on the lower horizontal scale. When G1 is zero, no flow can occur and when G1 is some maximum value, pressure P drops to zero. G2 is always large even at bearing lift-off and has no effect.

Feedback is stable. Note that if G1 becomes smaller for a given load on the rotor, P increases forcing G1 to increase. If G1 increases too much, P will decrease and so does the thrust bearing force. This reduces G1. At equilibrium, G1 reaches a steady state value and no longer changes. This all happens quickly enabling transient differential pressures to be measured.

This pressure transducer provides a means to determine the onset of inlet suction so it can be avoided. A fast-rising spike in differential pressure and its magnitude characterize suction. Avoiding this is important to obtain safe physiologic control with pulsating flow. Constant rpm LVADs have much less of a problem because their flow rates can be conservatively set without providing pulsating flow. The instant pressure transducer allows one to measure the pressure produced by the patient's beating heart (and the heart's state of recovery) independently of the pulsating pressure produced by the LVAD (which can be programmed at a differing frequency). Also, by maintaining systolic and diastolic differential pressures at preset reference values using cyclic rpm changes, one may provide a means for automatic flow rate control responsive to exercise. These methods are claimed in Goldowsky U.S. Pat. No. 6,527,699 for a totally magnetically suspended pump.

A screw pump operates by pumping fluid (blood) along the screw whose gross flow rate is theoretically independent of fluid viscosity. However, forward gross flow is reduced by back-leakage over the threads which depend on blood viscosity (for laminar flow past the threads). Laminar back flow can be made very small using long lands 28 instead of a smaller gap which has undesirable higher shear stress. A long length screw is advantageous to accomplish this (a long summed length of series lands) because space exists for a long screw in this preferred design. One may also elect to use a larger gap 24 with short thread lands to produce orifice flow. Orifice back flow is viscosity independent. Therefore, it is clear thrust bearing calibration can be made insensitive to blood viscosity (which is variable with hematocrit) for reliable long-term use.

The proposed thrust bearing's (force-deflection) characteristic is linear because a linear load line screw pump is employed as the pressure source (P versus Q is linear). However, rpm must be used to know which characteristic pump curve is in effect. This gives the final piece of real time information needed to calibrate rotor displacement. If a centrifugal type pump is used as the pressure source instead of a screw, then a plain central hole 14 is employed like in FIG. 1. The P versus Q curve is non-linear for centrifugal pumps and pressure rise depends on the square of rpm. A centrifugal type pump is usable (if employed on its negative slope) since its characteristic curves are insensitive to blood viscosity. A centrifugal pump is integrated in the instant invention by providing a plurality of small blades on the rotor in gap G1. This is shown in FIG. 16 of the Goldowsky U.S. Pat. No. 6,527,699 titled: “Magnetic Blood Pump”, to wash out the bearing gaps of the rotor.

In the event a large axial impact load is applied before the thrust bearing reacts, or if a steady load should exceed the thrust bearing's capability, the rotor will contact the end stators if even momentarily. These are typically titanium surfaces that can be coated with blood compatible diamond like carbon to provide low friction and high hardness to avoid damage. This, or a similar coating, should be employed for safety in the preferred embodiment of FIG. 1.

Axial blood pumps typically rotate at 5,000-20,000 rpm. Minimization of power consumption is an important objective. Power consumption by a hydrodynamic bearing can be reduced by reducing its diameter, shortening its length and reducing its radial gap. Additionally, blood shear stress is less in a smaller bearing, thus reducing blood damage and hemolysis.

Also, the length of a hydrodynamic bearing must be sufficiently long to provide cocking moment stability for the rotor. The greater the length, the greater the power consumption. Thus, as an alternative to a rotor having a single, relatively long hydrodynamic bearing, the rotor can be configured with two relatively short hydrodynamic bearings, one at each end of the rotor. The axial separation between the two bearings provides a large cocking moment capability.

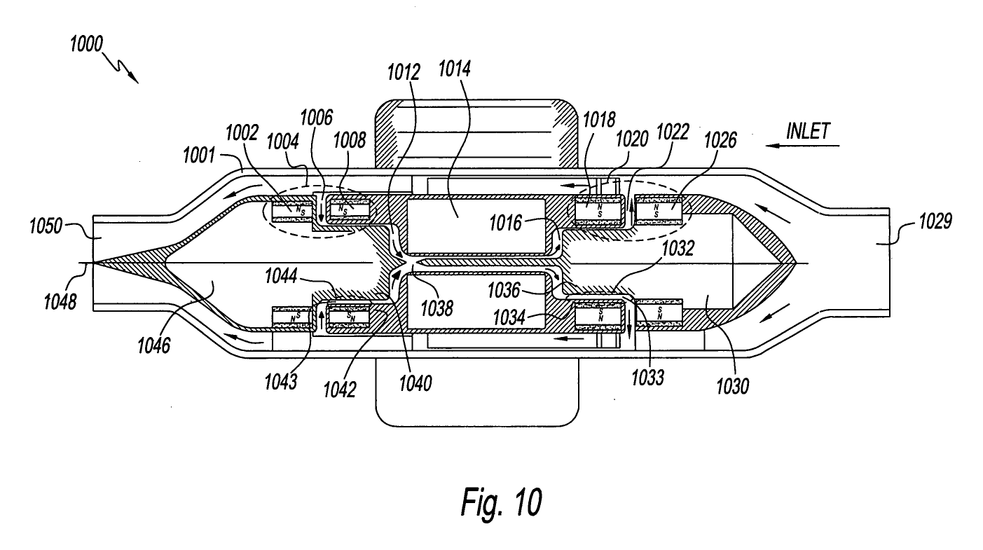

FIG. 10 is a longitudinal cross-section through a pump 1000. Pump 1000 includes a housing 1001 having an inlet 1029 and an outlet 1050. Within housing 1001, there is an inlet stator 1030, a rotor 1014, and an outlet stator 1046. Inlet stator 1030 and outlet stator 1046 are stationary with respect to housing 1001, and may be regarded as being part of housing 1001. Pump 1000 also includes, within housing 1001, two axial thrust bearings (i.e., thrust bearings 1004 and 1020, each of which is shown in a dashed oval), and two radial hydrodynamic bearings (i.e., hydrodynamic bearings 1044 and 1032, each of which is shown in a dashed oval). There is also an axis 1048 through pump 1000.

Rotor 1014 has a generally cylindrical shape centered about axis 1048, is rotatable about axis 1048, and includes a conduit 1038 along axis 1048. Since FIG. 10 is a cross-sectional view of pump 1000, rotor 1014 is shown in part (and labeled) near the upper central region of FIG. 10, but also shown in part (but not further labeled) near the lower central region of FIG. 10.

Thrust bearing 1004 is configured with a magnet 1002 and a magnet 1008 in an axially repulsive arrangement, acting across an axial gap, i.e., gap 1006. Magnets 1002 and 1008 are permanent magnets. Magnet 1002 is situated on outlet stator 1046, and magnet 1008 is situated on rotor 1014. Magnets 1002 and 1008, and gap 1006 each has a generally annular shape, i.e., like a doughnut or a lifesaver, centered about axis 1048. However, since FIG. 10 is a cross-sectional view of pump 1000, magnets 1002 and 1008, and gap 1006 are shown in part (and labeled) near the upper left of region rotor 1014, but also shown in part (but not further labeled) near the lower left region of rotor 1014. Thrust bearing 1004 axially, and passively, suspends an end of rotor 1014 that is near outlet stator 1046.

Thrust bearing 1020 is configured with a magnet 1018 and a magnet 1026 in an axially repulsive arrangement, acting across an axial gap, i.e., gap 1022. Magnets 1018 and 1026 are permanent magnets. Magnet 1018 is situated on rotor 1014, and magnet 1026 is situated on inlet stator 1029. Magnets 1018 and 1026, and gap 1022 each has a generally annular shape, i.e., like a doughnut or a lifesaver, centered about axis 1048. However, since FIG. 10 is a cross-sectional view of pump 1000, magnets 1018 and 1026, and gap 1022 are shown in part (and labeled) near the upper right region of rotor 1014, but also shown in part (but not further labeled) near the lower right region of rotor 1014. Thrust bearing 1020 axially, and passively, suspends an end of rotor 1014 near inlet stator 1030.

Hydrodynamic bearing 1044 includes a male portion 1040 and a female portion 1042. Male portion 1040 is also part of outlet stator 1046, and female portion 1042 is also part of rotor 1014. A radial gap, i.e., gap 1043, exists between a surface of male portion 1040 and a surface of female portion 1042. Hydrodynamic bearing 1044 and gap 1043 each has a generally cylindrical shape, centered about axis 1048. However, since FIG. 10 is a cross-sectional view of pump 1000, hydrodynamic bearing 1044 and gap 1043 are shown in part (and labeled) near the lower left region of rotor 1014, but also shown (but not further labeled) near the upper left region of rotor 1014. Hydrodynamic bearing 1044 radially, and passively, suspends the end of rotor 1014 near outlet stator 1046.

Hydrodynamic bearing 1032 includes a male portion 1036 and a female portion 1034. Male portion 1036 is also part of inlet stator 1030, and female portion 1034 is also part of rotor 1014. A radial gap, i.e., gap 1033, exists between a surface of male portion 1036 and a surface of female portion 1034. Hydrodynamic bearing 1032 and gap 1033 each has a generally cylindrical shape, centered about axis 1048. However, since FIG. 10 is a cross-sectional view of pump 1000, hydrodynamic bearing 1032 and gap 1033 are shown in part (and labeled) near the lower right region of rotor 1014, but also shown (but not further labeled) near the upper right region of rotor 1014. Hydrodynamic bearing 1032 radially, and passively, suspends the end of rotor 1014 near inlet stator 1030.

Hydrodynamic bearings 1044 and 1032 are spaced apart from one another, one at or near each end of rotor 1014. The axial separation between hydrodynamic bearings 1044 and 1032 provides a large cocking moment capability for rotor 1014. Also, hydrodynamic bearings 1044 and 1032 may include helical grooves to help pump blood therethrough.

In pump 1000, there is also (a) an axial gap, i.e., gap 1012, between outlet stator 1046 and rotor 1014, in the vicinity of axis 1048, and (b) an axial gap, i.e., gap 1016, between inlet stator 1030 and rotor 1014, in the vicinity of axis 1048. Gaps 1012 and 1016 have a generally annular shape, centered about axis 1048.

Gaps 1006 and 1022 are circumferential axial gaps. Gaps 1012 and 1016 are centerline axial gaps.

Paths of blood flow through pump 1000 are shown in FIG. 10, represented by a series of arrows. During operation of pump 1000, blood is drawn into inlet 1029 and around inlet stator 1030, through a space between an outer surface of inlet stator 1030 and an inner surface of housing 1001. The blood continues around rotor 1014, between an outer surface of rotor 1014 and the inner surface of housing 1001. When the blood arrives at gap 1006 it can flow in either of two paths. A majority portion of the blood flows around outlet stator 1046, between an outer surface of outlet stator 1046 and the inner surface of housing 1001, and exits via outlet 1050. However, some blood flows into gap 1006, through gaps 1043 and 1012, conduit 1038, gaps 1016 and 1033, and through gap 1022, and merges with the majority portion of the blood between the outer surface of rotor 1014 and the inner surface of housing 1001.

With regard to blood flow, gaps 1006, 1043, 1012, 1016, 1033, and 1022 are generally in series with each other. As such, all are washed out in series, with fresh blood, to eliminate thrombus. Differential pressure in pump 1000, in addition to the use of pumping grooves in hydrodynamic bearings 1032 and 1044, induces this washout.

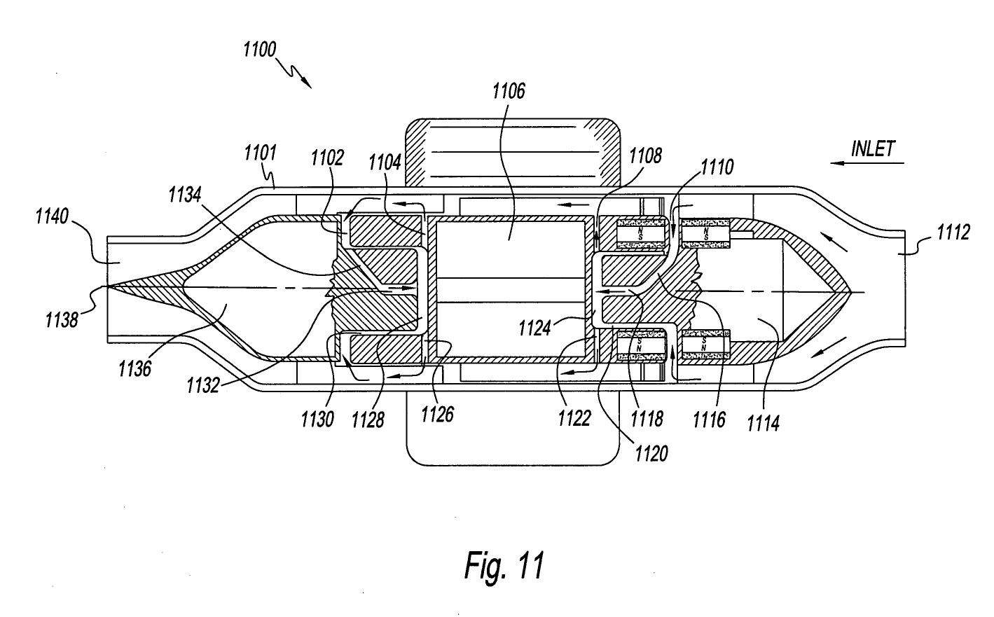

FIG. 11 is a longitudinal cross-section through a pump 1100 configured to provide for washing out gaps, using a flow of blood in a non-serial path. Pump 1100 includes a housing 1101 having an inlet 1112 and an outlet 1140. Within housing 1101 there is an inlet stator 1114, a rotor 1106, and an outlet stator 1136. Inlet stator 1114 and outlet stator 1136 are stationary with respect to housing 1101, and may be regarded as being part of housing 1101. There is also an axis 1138 through pump 1100. Rotor 1106 is rotatable about axis 1138.

Pump 1100 is similar, but not identical, to pump 1000. For example, although not labeled in FIG. 11, pump 1100 includes two axial thrust bearings and two radial hydrodynamic bearings, situated in pump 1100 similarly to the manner in which thrust bearings 1004 and 1020, and hydrodynamic bearings 1044 and 1032 are situated in pump 1000. However, in pump 1000, rotor 1014 includes conduit 1038, but in pump 1100, rotor 1106 does not include such a conduit. Moreover, as explained below, pump 1100 includes passages that are not present in pump 1000. Consequently, a path of blood flow through pump 1100 is different from the path of blood flow through pump 1000.

Pump 1100 includes (a) axial gaps, i.e., gaps 1102, 1128, 1124 and 1110, and (b) radial gaps, i.e., gaps 1130 and 1120. Gaps 1102 and 1110 are circumferential axial gaps. Gaps 1128 and 1124 are centerline axial gaps.

Inlet stator 1114 has a passage 1118 and a passage 1116. Passage 1118 is on axis 1138. Passage 1116 runs between passage 1118 and an exterior point on inlet stator 1114, in a vicinity of gap 1110. Although not shown in FIG. 11, inlet stator 1114 can be configured to include a plurality of passages 1116, each of which runs from a different exterior point on inlet stator 1114, in communication with gap 1110, and merges at passage 1118 with others of the plurality of passages 1116. For example, inlet stator 1114 could be configured with four passages 1116, at a spacing of 90 degrees.

Outlet stator 1136 has a passage 1132 and a passage 1134. Passage 1132 is on axis 1138. Passage 1134 runs between passage 1132 and an exterior point on outlet stator 1136, in communication with gap 1102. Although not shown in FIG. 11, outlet stator 1136 can be configured to include a plurality of passages 1134, each of which runs from a different exterior point on outlet stator 1136, in a vicinity of gap 1102, and merges at passage 1132 with others of the plurality of passages 1134. For example, outlet stator 1136 could be configured with four passages 1134, at a spacing of 90 degrees.

Rotor 1106 includes a plurality of radial passages, i.e., passages 1104, 1126, 1108 and 1122. Passage 1104 runs between gap 1128 and a first exterior point on rotor 1106. Passage 1126 runs between gap 1128 and a second exterior point on rotor 1106. Passage 1108 runs between gap 1124 and a third exterior point on rotor 1106. Passage 1126 runs between gap 1124 and a fourth exterior point on rotor 1106. Although FIG. 11 shows four passages 1104, 1126, 1108, and 1122, rotor 1106 can be configured with only one such passage, or with more than four such passages. The purpose of these passages is described below.

Paths of blood flow through pump 1100 are shown in FIG. 11, represented by a series of arrows. During operation of pump 1100, blood is drawn into inlet 1112 and around inlet stator 1114, through a space between an outer surface of inlet stator 1114 and an inner surface of housing 1101. When the blood reaches gap 1110, it can flow in either of two paths. A majority of the blood flows around rotor 1106, between an outer surface of rotor 1106 and the inner surface of housing 1101, and then around outlet stator 1136, between an outer surface of outlet stator 1136 and the inner surface of housing 1001, and exits via outlet 1140. However, some blood flows into gap 1110.

Blood that flows into gap 1110 can proceed thereafter into either gap 1120 or passage 1116. Blood in gap 1120 flows to gap 1124. Blood in passage 1116 flows to passage 1118. In an embodiment where inlet stator 1114 has a plurality of passages 1116, a portion of the blood from gap 1110 will flow through each of passages 1116, and merge into passage 1118. Blood from passage 1118 flows into gap 1124. From gap 1124, a portion of the blood flows through passage 1108, and a portion flows through passage 1122. The blood from passages 1108 and 1122 merges with the majority portion of the blood. Further downstream, although the majority of the blood proceeds on toward outlet 1140, some blood flows into gap 1102.

Blood that flows into gap 1102 can proceed thereafter into either gap 1130 or passage 1134. Blood in gap 1130 flows to gap 1128. Blood in passage 1134 flows to passage 1132. In an embodiment where outlet stator 1136 has a plurality of passages 1134, a portion of the blood from gap 1102 will flow through each of passages 1134, and merge into passage 1132. Blood from passage 1132 flows into passage 1126. From gap 1128, a portion of the blood flows through passage 1104, and a portion flows through passage 1126. The blood from passages 1104 and 1126 merges with the majority portion of the blood, and proceeds on toward outlet 1140.

Blood flowing through gaps 1102, 1130, 1128, 1124, 1120 and 1110, as described above, washes out these gaps. More specifically, the rotation of rotor 1106 centrifugally generates pressure (suction) directly across radial passages 1104 and across 1108, 1122 and 1126. The longer the radial passage, the greater the centrifugal pressure (or suction) generated. Passages 1104, 1126, 1108 and 1122 provide high centrifugal differential pressure to induce washout flows. For example, passage 1104 creates centrifugal suction that sucks in fresh blood through gap 1102. This washes out gaps 1102 and 1130. In addition, passage 1132 delivers this same suction to wash gap 1128. Similarly, passage 1108 sucks in fresh blood through gap 1110. This washes out gaps 1110 and 1120, and parallel flow washes gap 1124.

Passages 1132 and 1118 are of large enough diameter to have sufficiently low flow resistance. A key advantage of pump 1100 as compared to pump 1000 is that centrifugal suction can be much greater than pump differential pressure thereby giving more washout of the hydrodynamic bearings. Use of spiral pumping grooves to aid washing in these bearings is supplementary. Also, in pump 1100, since its hydrodynamic bearings are not washed in series with one another, washout flow in each is desirably increased, as compared to pump 1000, by a factor of two for the same pressure difference. In pump 1100, differential pressure generated across the axial flow pump is not the source of pressure for washing the bearings as was the case in FIG. 1.

Rotor axial position sensors may be included pumps 1000 and 1100, as shown in FIG. 1, to monitor rotor axial position for determining rotor differential pressure.

The techniques described herein are exemplary, and should not be construed as implying any particular limitation on the present invention. It should be understood that various alternatives, combinations and modifications could be devised by those skilled in the art. The present invention is intended to embrace all such alternatives, modifications and variances that fall within the scope of the appended claims.

What is claimed is:

1. A blood pump comprising:
(a) a housing;
(b) a pump rotor within said housing, wherein said pump rotor has a first end and a second end, and an axis of rotation;
(c) a first axial thrust bearing across a first axial gap, between said first end and said housing, that axially suspends said first end;
(d) a second axial thrust bearing across a second axial gap, between said second end and said housing, that axially suspends said second end;
(e) a first radial hydrodynamic bearing that radially suspends said first end; and
(f) a second radial hydrodynamic bearing that radially suspends said second end.

2. The blood pump of claim 1,
wherein said first axial thrust bearing comprises magnets in an axially repulsive arrangement acting across said first axial gap, and
wherein said second axial thrust bearing comprises magnets in a repulsive arrangement acting across said second axial gap.

3. The blood pump of claim 2, where an axial position of said pump rotor is measured to obtain rotor differential pressure.

4. The blood pump of claim 1, wherein said first and second axial thrust bearings and said first and second radial hydrodynamic bearings passively suspend said pump rotor in said housing so that said pump rotor does not contact said housing.

5. The blood pump of claim 1, wherein said pump rotor has a series fluid conduit along said axis connecting said hydrodynamic bearings.

6. The blood pump of claim 5, wherein, during operation of said blood pump, blood flows along a path that includes said first axial gap and said conduit.

7. The blood pump of claim 6, wherein said blood washes said first axial gap to prevent thrombus therein.

8. The blood pump of claim 1, wherein said pump rotor has a radial passage that, during operation of said blood pump, generates centrifugal pressure that washes out said first axial gap.

9. The blood pump of claim 1, wherein said pump rotor has a radial passage that, during operation of said blood pump, generates centrifugal pressure that washes out a gap in said first radial hydrodynamic bearing.

10. The blood pump of claim 1, wherein said pump rotor has a radial passage that, during operation of said blood pump, generates centrifugal pressure that washes out said first axial gap and said first radial hydrodynamic bearing.
